(19) United States
(12) Patent Application Publication
(10) Pub. No.: US 2013/0317234 A1
(43) Pub. Date: Nov. 28, 2013

(54) Process for Preparation of Intermediates of Bendamustine

(71) Applicants: Manik Reddy Pullagurla, Hyderabad (IN); Jagadeesh Babu Rangisetty, Hyderabad (IN); S. I. Davis Presley, Thiruninravur (IN); Radha Nagarapu, Hyderabad (IN)

(72) Inventors: Manik Reddy Pullagurla, Hyderabad (IN); Jagadeesh Babu Rangisetty, Hyderabad (IN); S. I. Davis Presley, Thiruninravur (IN); Radha Nagarapu, Hyderabad (IN)

(73) Assignee: BIOPHORE INDIA PHARMACEUTICALS PVT. LTD, Hyderabad, AP (IN)

(21) Appl. No.: 13/877,932

(22) PCT Filed: Jul. 14, 2011
(86) PCT No.: PCT/IN2011/000471

§ 371 Date: Apr. 5, 2013

(30) Foreign Application Priority Data
Jul. 15, 2010 (IN) 2005/CHE/2010

(51) Int. Cl. C07D 235/16

(52) U.S. Cl. CPC C07D 235/16
USPC 548/310.1

(57) ABSTRACT

The present invention relates to a process for the preparation of 4-{5-{Bis-(2-hydroxyl-ethyl)-amino}-1-methyl-1H-Benzoimidazol-2yl}-butyric acid alkyl ester of formula IV, a key intermediate in the process for the preparation of Bendamustine HCl (I)

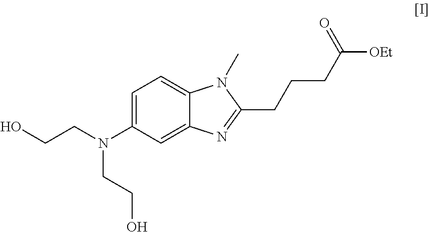 [SMILES: CCOC(=O)CCCc1nc2cc(N(CCO)CCO)ccc2n1C]

### FIELD OF INVENTION

The present invention relates to a process for the preparation of 4-{5-[Bis-(2-hydroxyl-ethyl)-amino]-1-methyl-1H-Benzoimidazol-2yl}-butyric acid ethyl ester of formula IV, a key intermediate in the process for the preparation of Bendamustine HCl (I).

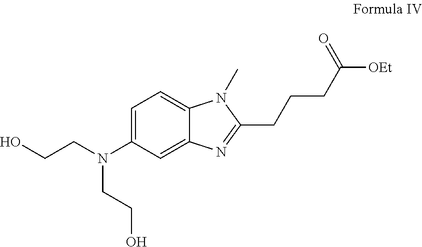 [SMILES: CCOC(=O)CCCc1nc2cc(N(CCO)CCO)ccc2n1C]

### BACKGROUND OF THE INVENTION

Bendamustine (I) acts as an alkylating agent and is used to treat chronic lymphocytic leukemia, Hodgkins disease, non-Hodgkin's lymphoma, multiple myeloma and breast cancer.

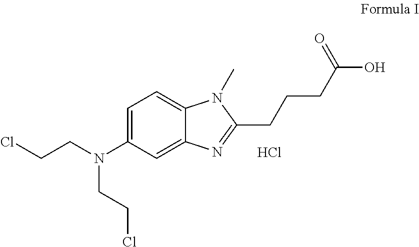 [SMILES: Cn1c(CCCC(=O)O)nc2cc(N(CCCl)CCCl)ccc21 ; Cl]

Bendamustine was originally reported by Ozegowaski and Krebs in Journal fur Praktische Chemie 20, 1963, pp 178-186 and was available from 1971 to 1992 in Germany under the name Cytostasan. Since that time, it has been marketed in Germany under the tradename Ribomustin. Recently Bendamustine was approved by USFDA for the treatment of indolent b-cell non-Hodgkin's lymphoma and marketed as Treanda.

Journal fur Praktische Chemie 20, 1963, pp178-186 described the synthesis of Bendamustine via the synthesis of intermediate of Formula IV by alkylation reaction of Formula III in presence of ethylene oxide.

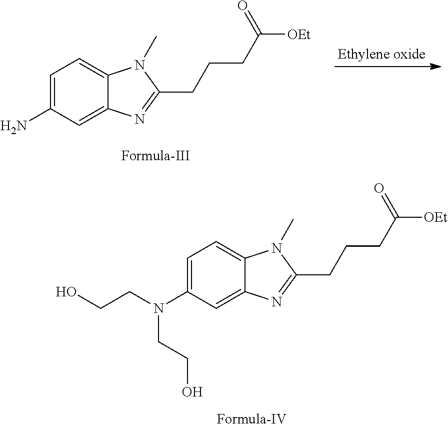 [SMILES: CCOC(=O)CCCc1nc2cc(N)ccc2n1C ; CCOC(=O)CCCc1nc2cc(N(CCO)CCO)ccc2n1C]

DD134727 discloses a method of preparing intermediates of Bendamustine and also discloses the alkylation reaction of Formula III by ethylene oxide.

DD159877 discloses the conversion of compound of Formula IV to Bendamustine in presence of thionyl chloride followed by hydrolysis under acidic conditions at 95 ° C.

WO2009120386 discloses novel crystalline forms of Bendamustine Hydrochloride and also describes a process to obtain Bendamustine from compound of formula IV in presence of thionyl chloride followed by hydrolysis under HCl conditions at 85-90° C. The final isolation is also described by distillation of water followed by crystallization.

WO2010042568 describes a novel process for the preparation of Bendamustine starting from 1-methylamino-2,4-dinitrobenzene.

### SUMMARY OF THE INVENTION

Most of the prior art processes for the synthesis of Bendamustine essentially require the formation of compound of Formula IV, a crucial intermediate via the alkylation reaction mediated by ethylene oxide. Use of ethylene oxide being a highly explosive chemical at an industrial scale is very dangerous. The reaction work ups for eliminating ethylene oxide at a small scale is possible with water base treatment as reported in the art. However, the present inventors observed that the water base treatment at a large-scale synthesis is not effective in removing considerable amounts of ethylene oxide as impurity in the reaction product IV. This results in the degradation of the compound IV, thereby decreasing the yield of Bendamustine considerably at industrial scale.

Therefore there is a need in the art for the production of Bendamustine, in a safe and viable route applicable at an industrial scale.

The invention further describes a convenient route with less tedious work up for the hydrolysis of compound IV to obtain substantially pure Bendamustine.

Thus we propose a safe and convenient route for the synthesis of Intermediate IV without the use of ethylene oxide, which is subsequently hydrolysed to obtain substantially pure Bendamustine Ha.

### DETAILED DESCRIPTION OF THE INVENTION

The aim of the present invention is to obtain the desired compound with improved process, safety, yield and purity.

One object of the invention provides a process for the preparation of compound of formula IV by the alkylation of compound of formula III by 2-haloethanol in the presence of a base and optionally in the presence of a metal halide.

The reaction can be carried out in the presence of inorganic bases such as sodium carbonate, potassium carbonate, sodium bicarbonate, sodium hydroxide: potassium hydroxide and the like.

The haloalkanols employed are 2-chloroethanol, 2-bromoethanol and 2-iodoethanol and preferably 2-chloroethanol.

The reaction can be carried out from 25° C. to 125° C. and preferably at 60-70 ° C. The reaction time of the process is from 2 to 15 hours and dependent on the temperature of the reaction. It has been found that at higher temperatures the reaction is completed within 2 h and at low temperatures it takes about 15 h.

Subsequent conversion of formula IV to Bendamustine was carried out in thionyl chloride followed by hydrolysis with conc. HCl.

It has been surprisingly found that the hydrolysis reaction when performed at 55-65° C. and reducing the amount of conc. HCl to <10 volumes.

The invention is schematically represented as:

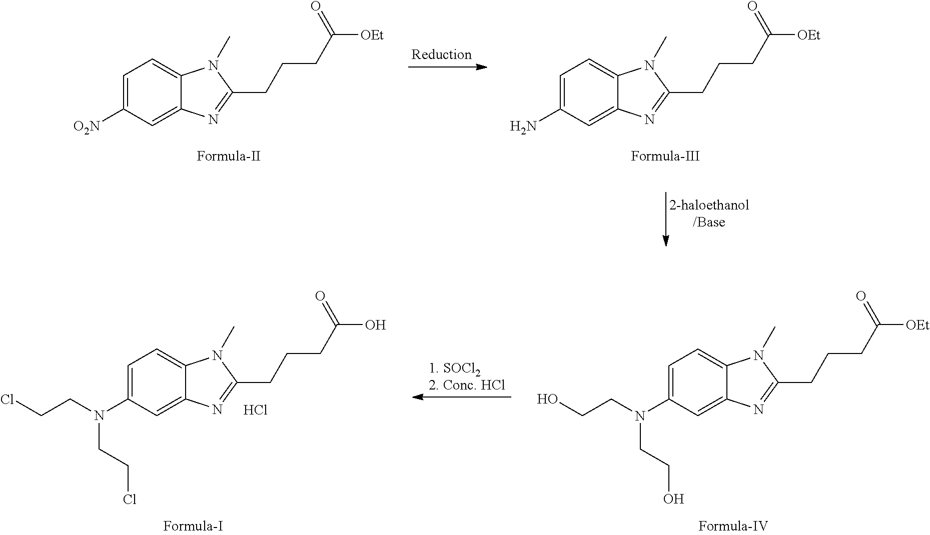 [SMILES: CCOC(=O)CCCc1nc2cc([N+](=O)[O-])ccc2n1C ; CCOC(=O)CCCc1nc2cc(N)ccc2n1C ; Cn1c(CCCC(=O)O)nc2cc(N(CCCl)CCCl)ccc21 ; Cl ; CCOC(=O)CCCc1nc2cc(N(CCO)CCO)ccc2n1C]

The following examples further illustrate the present invention.

### EXAMPLE-I

Preparation of Ethyl 4-(5-Amino-1-Methyl-1H-Benzo[d]Imidazol-2-yl)Butanoate (Formula-III)

To a clean dry flask were charged Iron powder (85 g), Conc. HCl (12.5 ml), and 625 ml of methanol and stirred for 5 minutes at room temperature. The contents were heated to 60-65° C. and maintained for 2 hours. At that temperature ammonium chloride solution was charged and maintained for 15 minutes and subsequently cooled to RT. Compound II (80 g) was added and then the reaction mass was maintained at 60-65° C. for 2 h. The mass was cooled to room temperature, filtered and distilled. To the residue was charged water and The pH adjusted to 7-8 using sodium bicarbonate solution. The Aqueous layer is extracted with ethyl acetate and the organic layer is distilled out completely to the give the title compound as a solid (60 g).

### EXAMPLE-II

Preparation of Ethyl 4-(5-(bis(2-Hydroxyethyl)Amino)-1-Methyl-1H-Benzo[d]-Imidazol-2-yl)Butanoate (Formula-IV)

To a clean dry flask were charged compound of formula III (20 g), sodium carbonate (16.24 g), sodium Iodide (10.6 g) and 80 ml of 2-chloroethanol. The mixture was stirred for 5 minutes at room temperature and the reaction mass was heated to 65-70° C. and maintained for 8-12 hours. The mass was cooled to room temperature and pH adjusted to 1.0 using 6N HCl. The aqueous layer was extracted with ethyl acetate and the aqueous layer pH is adjusted to 8 to 9 using sodium carbonate solution. The Aqueous layer is extracted with dichloromethane and is distilled out completely and the solid obtained which is then purified in ethyl acetate to give 10 g of the title compound.

### EXAMPLE-III

### Preparation of Bendamustine Hydrochloride (Formula-I)

To a compound of formula IV (10 g) in dichloromethane (100 ml), thionyl chloride (20 ml) is charged slowly at 0-5° C. and then allowed to reflux. The reaction is distilled to dryness and then 100 ml conc. HCl is charged to the reaction. The temperature is raised to 80-90 ° C. and maintained for 6-8 h. The mass is treated with activated carbon, filtered and distilled to give the title product, which is washed with acetone (100 ml).

### EXAMPLE-IV

### Preparation of Bendamustine Hydrochloride (Formula-I)

To a compound of formula IV (10 g) in dichloromethane (100 ml), thionyl chloride (20 ml) is charged slowly at 0-5 ° C. and then allowed to raise to 40-45° C. The reaction is distilled to dryness and then 30 ml conc. HCl is charged to the reaction. The temperature is raised to 55-60 ° C. for 6-8 h. The mass is treated with activated carbon and filtered. The mass is then cooled to give the Bendamustine Hydrochloride, which was recrystallized from acetone and water mixture to give Bendamustine Hydrochloride Monohydrate as a white solid.

We claim:

1. A process for the preparation of compound of formula IV by the alkylation of compound of formula III by a 2-haloethanol in the presence of a base.

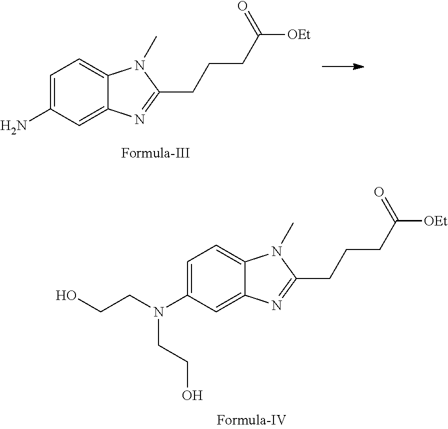 [SMILES: CCOC(=O)CCCc1nc2cc(N)ccc2n1C ; CCOC(=O)CCCc1nc2cc(N(CCO)CCO)ccc2n1C]

2. The process according to claim 1 wherein 2-haloethanol is 2-chloroethanol and the base employed is sodium carbonate

3. The process according to claim 1 wherein the reaction is carried out at a temperature of 65-70° C. for 8-12 h.

4. A process for the preparation of Bendamustine by the hydrolysis of compound of Formula IV, wherein the process involves
a) addition of thionyl chloride to Formula IV at a temp of 0-5 ° C.
b) raising the reaction temperature to 40-45 ° C.
c) distilling the reaction mass to dryness and adding conc. HCl (3 volumes) followed by heating to 55-65 ° C. for 6-8 h
d) cooling the reaction mass followed by filtering to obtain Bendamustine hydrochloride.
